(19) United States
(12) Patent Application Publication
(10) Pub. No.: US 2022/0406464 A1
(43) Pub. Date: Dec. 22, 2022

(54) PREDICTION METHOD AND SYSTEM OF LOW BLOOD PRESSURE

(71) Applicant: ACUSENSE BIOMEDICAL TECHNOLOGY CO., LTD., Tainan City (TW)

(72) Inventors: Hsiang-Wei HU, Tainan City (TW); Chih-Hao LIU, Tainan City (TW); Chi-Hin UN, Tainan City (TW); Kuan-Yu CHEN, Tainan City (TW); Jiun-Yi YANG, Tainan City (TW)

(21) Appl. No.: 17/500,921

(22) Filed: Oct. 13, 2021

(30) Foreign Application Priority Data
Jun. 18, 2021 (TW) 110119774

(51) Int. Cl. G16H 50/30; G16H 20/40; G06N 5/02

(52) U.S. Cl. CPC G16H 50/30; G16H 20/40; G06N 5/02; A61M 2230/30; A61M 1/1603

(57) ABSTRACT

A prediction method and system of a low blood pressure is provided, including: obtaining a plurality of feature sequence values; selecting two of the feature sequence values from the feature sequence values according to a time ratio relationship; calculating a relation coefficient according to the selected two feature sequence values by a weighting process; repeating to select the new feature sequence values and the corresponding relation coefficient and to assign the new feature sequence values and the relation coefficient into the input group until the feature sequence values conforming to the time ratio relationship are traversed; and obtaining a training result by substituting the input group into a low blood pressure prediction model.

### CROSS-REFERENCE TO RELATED APPLICATION

This non-provisional application claims priority under 35 U.S.C. § 119(a) to Patent Application No. 110119774 filed in Taiwan, R.O.C. on Jun. 18, 2021, the entire contents of which are hereby incorporated by reference.

### BACKGROUND

### Technical Field

The disclosure relates to a prediction method and system of physiological characteristics, in particular to a prediction method and system of a low blood pressure.

### Related Art

During hemodialysis, patients may face many problems. Especially when patients have a low blood pressure, besides the need to terminate the course of hemodialysis, other side effects may occur. At present, the low blood pressure is detected under the real-time care of medical workers. However, for medical centers, most of the manpower arrangements are a single medical worker taking care of multiple patients. Therefore, when the patient has a low blood pressure during hemodialysis, the medical worker cannot know it immediately.

### SUMMARY

In view of this, in some examples, a prediction method of a low blood pressure includes steps as follows. A plurality of feature sequence values are obtained. Two feature sequence values are selected from the feature sequence values according to a time ratio relationship. A relation coefficient is calculated according to the selected two feature sequence values by a weighting process. To select the new feature sequence values and the corresponding relation coefficient and to assign the new feature sequence values and the relation coefficient into the input group are repeated until the feature sequence values conforming to the time ratio relationship are traversed. A training result is obtained by substituting the input group into a low blood pressure prediction model. The prediction method of a low blood pressure can provide more effective data, thereby improving training of the low blood pressure prediction model.

In some examples, the step of obtaining the feature sequence values includes the following content. A current cycle and a past cycle are set. The current cycle and the past cycle have a time corresponding relationship. The feature sequence value obtained in the current cycle is used as a first eigenvalue. The feature sequence value obtained in the past cycle is used as a second eigenvalue. Each of the first eigenvalues corresponds to the second eigenvalue according to the time corresponding relationship.

In some examples, the step of calculating the relation coefficient by the weighting process includes the following content. A processor obtains a corresponding first relation coefficient according to the first eigenvalues. The processor obtains a corresponding second relation coefficient according to the second eigenvalues. The processor adds the corresponding two first eigenvalues to the input group according to the first relation coefficient. The processor obtains the second relation coefficient corresponding to the selected first relation coefficient according to the time corresponding relationship. The processor adds the corresponding two second eigenvalues obtained according to the second relation coefficient to the input group.

In some examples, the weighting process includes a difference operation, a quotient operation, a multinomial coefficient operation, a trend calculation, an autoregressive model or a moving average model.

In some examples, the step of obtaining the training result by substituting the input group into the low blood pressure prediction model includes the following content. The feature sequence values of the input group are divided into a training group, a verification group and a test group. The training result corresponding to the training group is obtained by inputting the training group into the low blood pressure prediction model.

In some examples, the step of obtaining the training result corresponding to the training group by inputting the training group into the low blood pressure prediction model includes the following content. A result to be verified is obtained by substituting the test group into the training result and the low blood pressure prediction model. A verification result is obtained according to the verification group and the result to be verified.

In some examples, the step of repeating to select the new feature sequence values and the corresponding relation coefficient and to assign the new feature sequence values and the relation coefficient into the input group includes the following content. If the relation coefficient is more than a low blood pressure threshold, the relation coefficient and the two feature sequence values corresponding to the relation coefficient are assigned into an input group. To select the new feature sequence values and to assign the new feature sequence values into the input group are repeated until all the feature sequence values are traversed.

In some examples, the feature sequence values include heart rate variation coefficient, heart rate mean, blood oxygen variation coefficient, blood oxygen mean, diastolic blood pressure, mean arterial pressure, pulse, systolic blood pressure, pulse pressure, blood flow velocity, cumulative exchange blood volume, loop arterial pressure, blood temperature, bicarbonate concentration, electrical conductivity, dialysate flow velocity, anticoagulant maintenance dose, sodium ion, target sodium ion concentration, artificial kidney transmembrane pressure, machine set temperature, dehydration rate, dehydration time, current total dehydration, loop venous pressure or combinations thereof.

In some examples, the low blood pressure prediction model includes a LightGBM model, an Xgboost model, a Linear Regression model, a Random Forest model, a 1DCNN model, a DNN model, a LSTM model or a GRU model.

In some examples, a prediction system of a low blood pressure includes a data collection end and a server. The data collection end receives a plurality of feature sequence values. The server is connected to the data collection end. The server has a processor, a communication unit and a storage unit. The communication unit is connected to the data collection end. The communication unit transmits the feature sequence values. The storage unit stores a feature processing program and a low blood pressure prediction model. The processor executes the feature processing program. The processor obtains the feature sequence values through the transmission unit. The processor selects two feature sequence values according to a time ratio relationship. The processor calculates a relation coefficient according to the selected two feature sequence values by a weighting process. The processor repeats to select the new feature sequence values and the corresponding relation coefficient and to assign the new feature sequence values and the relation coefficient into the input group until the feature sequence values conforming to the time ratio relationship are traversed. The processor obtains a training result by substituting the input group into the low blood pressure prediction model.

In the prediction method and system of a low blood pressure, the relation coefficients are obtained according to the various feature sequence values of the object, and the feature sequence values and the relation coefficients are assigned by the feature processing program. The feature processing program substitutes the assignment result into the low blood pressure prediction model, thereby obtaining the corresponding training result of the object. The feature processing program can more accurately predict blood pressure variations of the object through the training result and the low blood pressure prediction model.

### BRIEF DESCRIPTION OF THE DRAWINGS

FIG. 1 is a schematic diagram showing architecture of a system according to one example;

FIG. 2 is a schematic diagram showing a training processing flow of predicting a low blood pressure according to one example;

FIG. 3A is a schematic diagram of obtaining feature sequence values and relation coefficients in a current cycle according to one example;

FIG. 3B is another schematic diagram of obtaining feature sequence values and relation coefficients according to one example;

FIG. 4A is a schematic diagram showing a training processing flow of predicting a low blood pressure according to one example;

FIG. 4B is a broken line graph of a feature sequence value and a relation coefficient corresponding to Table 2 according to one example;

FIG. 4C is another broken line graph of a feature sequence value and a relation coefficient corresponding to Table 2 according to one example;

FIG. 5 is a schematic diagram showing an operation flow of a training group, a verification group and a test group according to one example; and

FIG. 6 is a schematic diagram of selecting input groups in the current cycle and the past cycle according to one example.

### DETAILED DESCRIPTION

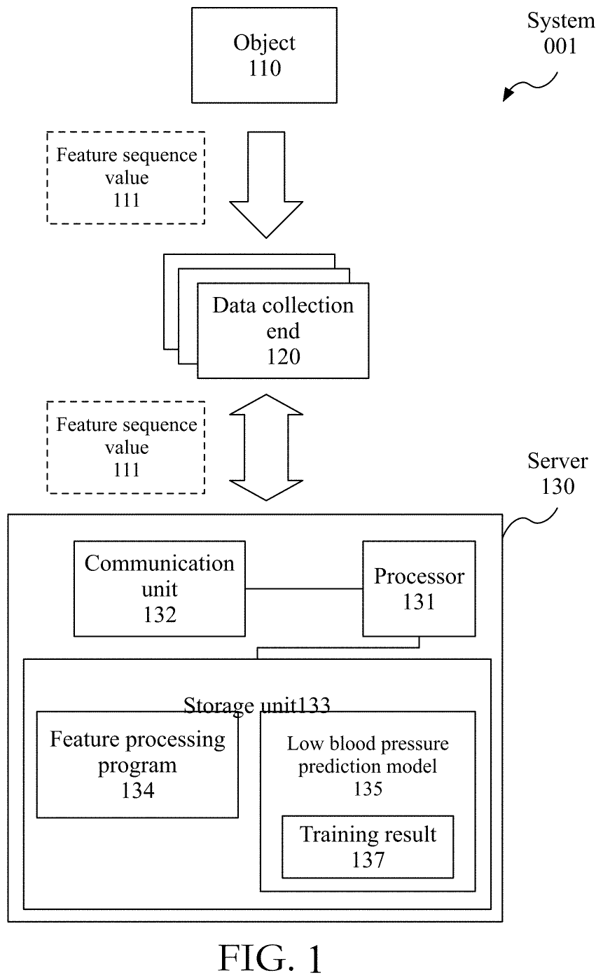

FIG. 1 is a schematic diagram showing architecture of hardware according to one example. A prediction system 001 of a low blood pressure in one example includes at least one data collection end 120 and a server 130. The data collection end 120 is connected to the server 130, and the connection mode includes at least a wired network connection, a wireless network connection or a cable connection. The data collection end 120 may be, but is not limited to, a wearable device, and the data receiving terminal may also be a medical facility, for example, a hemodialysis machine and a peritoneal dialysis machine. The data collection end 120 receives a plurality of feature sequence values 111 of an object 110. The object 110 may be a patient, a medical facility or a combination thereof. In other words, the object 110 may be a detected patient and a medical facility to which the patient belongs. Although a single object 110 is shown in FIG. 1, it actually and generally refers to the combination of the patient and the medical facility. The data collection end 120 may obtain the feature sequence values 111 in real time, and may also transmit the plurality of feature sequence values 111 to the server 130 at one time.

The feature sequence values 111 include heart rate variation coefficient, heart rate mean, blood oxygen variation coefficient, blood oxygen mean, diastolic blood pressure, mean arterial pressure, pulse, systolic blood pressure, pulse pressure, blood flow velocity, cumulative exchange blood volume, loop arterial pressure, blood temperature, bicarbonate concentration, electrical conductivity, dialysate flow velocity, anticoagulant maintenance dose, sodium ion, target sodium ion concentration, artificial kidney transmembrane pressure, machine set temperature, dehydration rate, dehydration time, current total dehydration, loop venous pressure or combinations thereof.

As for the data collection end 120, when the object 110 starts hemodialysis, the data collection end 120 obtains the feature sequence values 111 of the object 110 at specific intervals. The feature sequence value 111 may be not only a single parameter, but also a set of multiple parameters. For example, the feature sequence value 111 may be the systolic blood pressure as the single parameter. The data collection end 120 may also select both the systolic blood pressure and the diastolic blood pressure as the feature sequence value 111. If a processing time of one hemodialysis process is 4 hours and the data collection end 120 can collect the feature sequence value 111 of the object 110 every 10 minutes, then the data collection end 120 can collect 24 (4*60÷10) pieces of data. All the data collected are the feature sequence values 111. The data collection end 120 is not limited to the aforementioned time cycle of ten minutes.

The number of the data collection ends 120 is determined according to the environment at the scene. Taking the wearable device and the hemodialysis machine as an example, the wearable device may detect a pulse, a blood pressure or a body temperature of the object 110. Therefore, the data collection end 120 may collect the feature sequence value 111 of the pulse, the blood pressure or the body temperature. The hemodialysis machine may detect the feature sequence value 111, such as blood oxygen, blood flow velocity and sodium ion concentration, of the object 110.

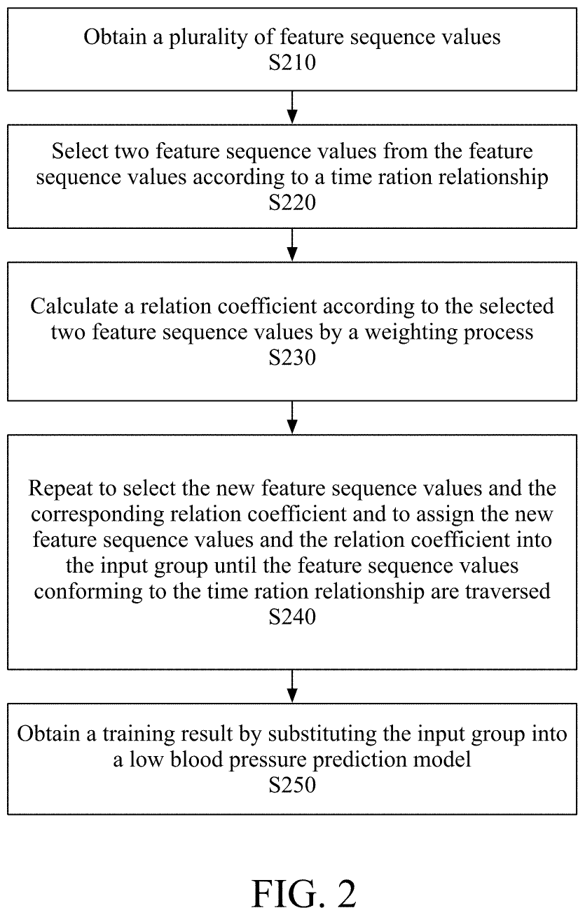

The server 130 has a processor 131, a communication unit 132 and a storage unit 133. The processor 131 is electrically connected to the communication unit 132 and the storage unit 133. The communication unit 132 is connected to the server 130, and the communication unit 132 transmits the feature sequence values 111. The storage unit 133 stores a feature processing program 134 and a low blood pressure prediction model 135. The processor 131 executes the feature processing program 134 and the low blood pressure prediction model 135. The feature processing program 134 calculates and assigns the obtained feature sequence values 111, and substitutes the obtained result into the low blood pressure prediction model 135. The server 130 may be a local terminal connected to the data collection end 120, or may be connected to the data collection end 120 through a remote network. FIG. 2 is a schematic diagram showing a training processing flow of predicting a low blood pressure according to one example. The training processing method of predicting a low blood pressure according to this example includes steps as follows.

Step S210: A plurality of feature sequence values are obtained.

Step S220: Two feature sequence values are selected from the feature sequence values according to a time ratio relationship.

Step S230: A relation coefficient is calculated according to the selected two feature sequence values by a weighting process.

Step S240: To select the new feature sequence values and the corresponding relation coefficient and to assign the new feature sequence values and the relation coefficient into the input group are repeated until the feature sequence values conforming to the time ratio relationship are traversed.

Step S250: A training result is obtained by substituting the input group into a low blood pressure prediction model.

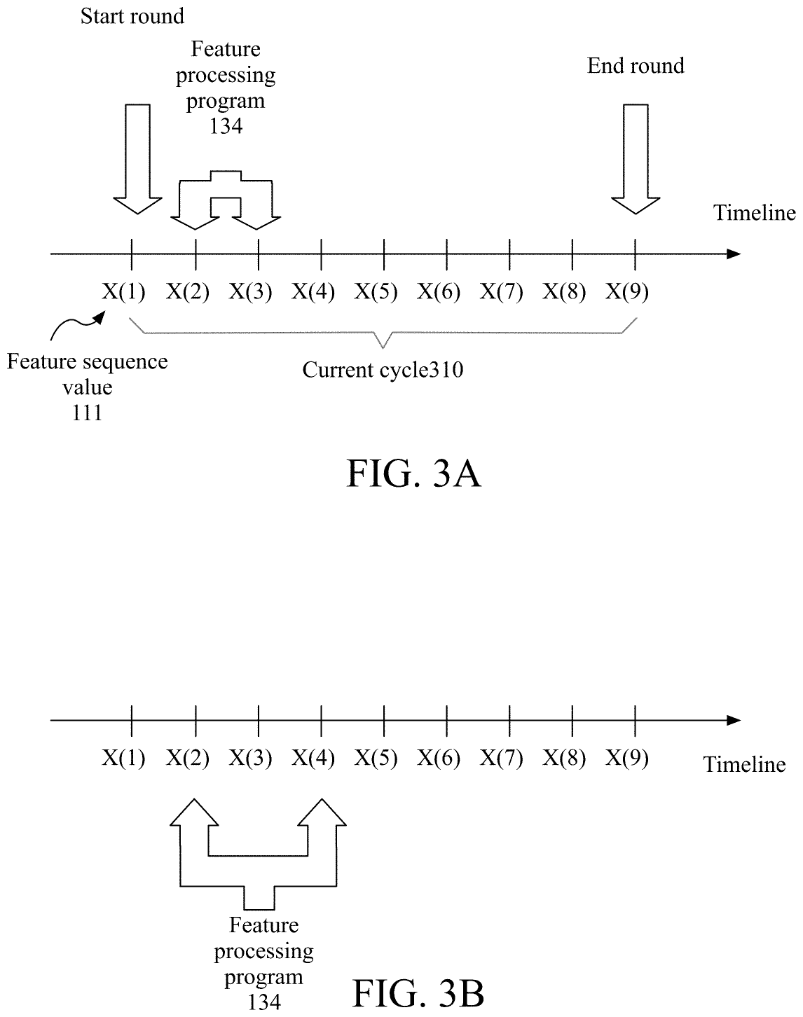

The data collection end 120 obtains the plurality of feature sequence values 111 of an object 110. In order to clearly explain the obtainment period of the feature sequence value 111, the period of hemodialysis is taken as the same obtainment period. The current obtainment period of the data collection end 120 is referred to as a current cycle 310, with reference to FIG. 3A.

In FIG. 3A, the feature sequence value 111 is expressed as X(m). X is the current cycle 310, and m is the number of sampling rounds of the feature sequence value 111 (may also be the sampling time point). For example, when m is “1”, X(m) represents the feature sequence value 111 sampled in the “1^{st}” round. When m is “10”, X(m) represents the feature sequence value 111 sampled in the “10^{th}” round. If m∈{1. 2 . . . , 8, 9, 10}, then the feature sequence value X(1) is information obtained at the start of the hemodialysis, and the feature sequence value X(10) is information obtained at the end of the hemodialysis.

Next, the feature processing program 134 selects two feature sequence values from the current cycle 310 according to a time ratio relationship. The time ratio relationship is a range of the interval of sampling sequence feature sequence values. The time ratio relationship is expressed in integers. If the time ratio relationship is 1, it means that the feature processing program 134 samples two sets of adjacent feature sequence values, with reference to FIG. 3A.

Taking FIG. 3A as an example, there are 10 feature sequence values X(1)-X(10) in the current cycle 310. It is assumed that the feature processing program 134 takes the feature sequence value X(1) as the calculation of the relation coefficient in the first round and selects another feature sequence value X(0) in a manner that the time ratio relationship is “1”. Since the feature sequence value X(0) does not exist, the feature processing program 134 does not calculate the relation coefficient of this round. In the third round, the feature processing program 134 calculates the relation coefficient R(3,2) according to X(2) and X(3) by a weighting process. In the calculation of the relation coefficient in the third round, the feature processing program 134 takes the feature sequence value X(3) as the basis and selects another feature sequence value X(2). The feature processing program 134 obtains another relation coefficient R(4,3) in the calculation of the 4th round. In the other rounds, the new feature sequence values are selected and the relation coefficient is calculated in this manner.

If the time ratio relationship is “2”, then the feature processing program 134 obtains two feature sequence values at an interval of a group of feature sequence values, with reference to FIG. 3B. The manner of selecting the two feature sequence values is to respectively obtain a feature sequence value X(n) and a feature sequence value X(n−1) according to the time ratio relationship based on the current feature sequence value X(n). If n is “0”, then the feature processing program 134 does not perform the weighting process. Similarly, when the feature sequence value is X(n+1), then the feature processing program 134 selects the feature sequence values X(n+1) and X(n) to perform the operation of the relation coefficient.

The feature processing program 134 calculates the corresponding relation coefficient R(a,b) according to the selected two feature sequence values 111 by the weighting process. a and b respectively represent the number of sampling rounds corresponding to the feature sequence value. The type of the weighting process includes a difference operation, a quotient operation, a multinomial coefficient operation, a trend calculation, an autoregressive model or a moving average model. Taking the difference operation as an example, the feature processing program 134 selects the feature sequence values X(2) and X(1), and performs subtraction on the two feature sequence values (X(2)−X(1)) to obtain the relation coefficient R(2,1). In addition, the weighting process can also calculate the relation coefficient through the quotient of the two feature sequence values.

The feature processing program 134 sequentially selects the feature sequence values 111 according to the time ratio relationship until the current round 310 ends. The feature processing program 134 obtains a training result 136 by substituting the selected feature sequence values 111 and the corresponding relation coefficient into the low blood pressure prediction model 135. The type of the low blood pressure prediction model 135 may be, but is not limited to, a LightGBM (Light Gradient Boosting Decision Tree) model, an Xgboost model, a Linear Regression model, a Random Forest model, a One Dimensional Convolutional Neural Network (1D CNN) model, a Deep Neural Network (DNN) model, a Long Short Term Memory Networks (LSTM) model or a Gated Recurrent Unit (GRU) model.

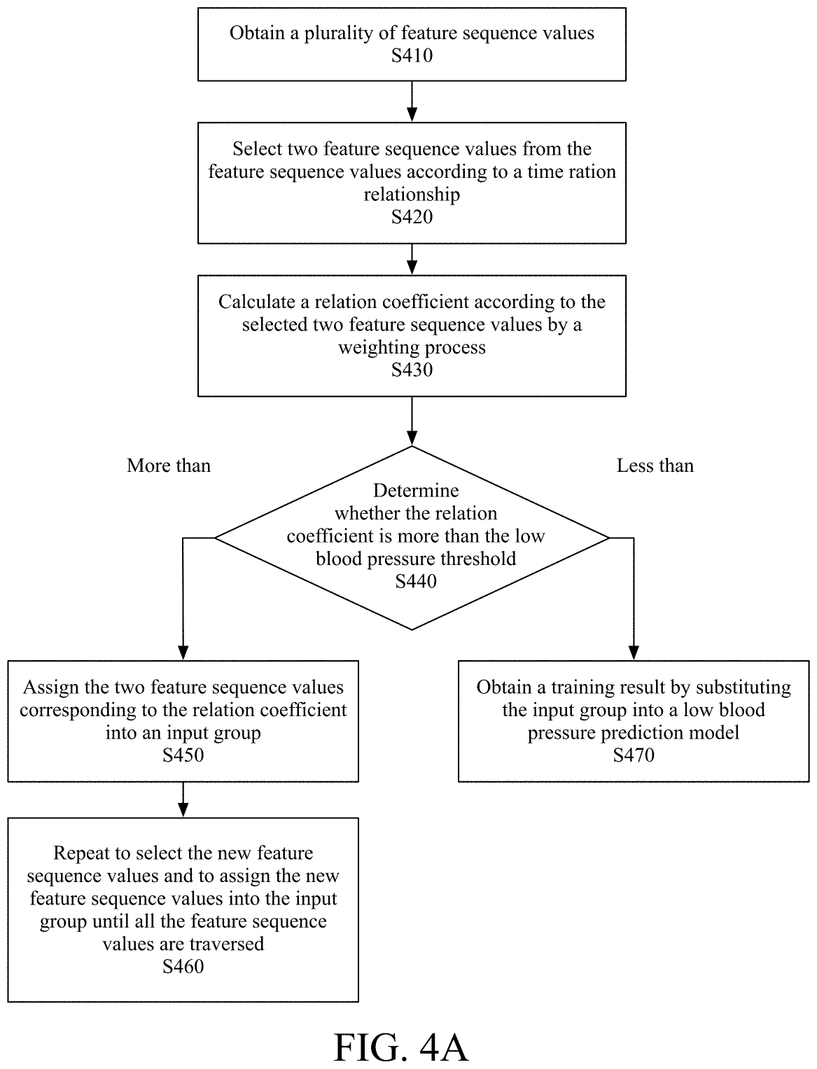

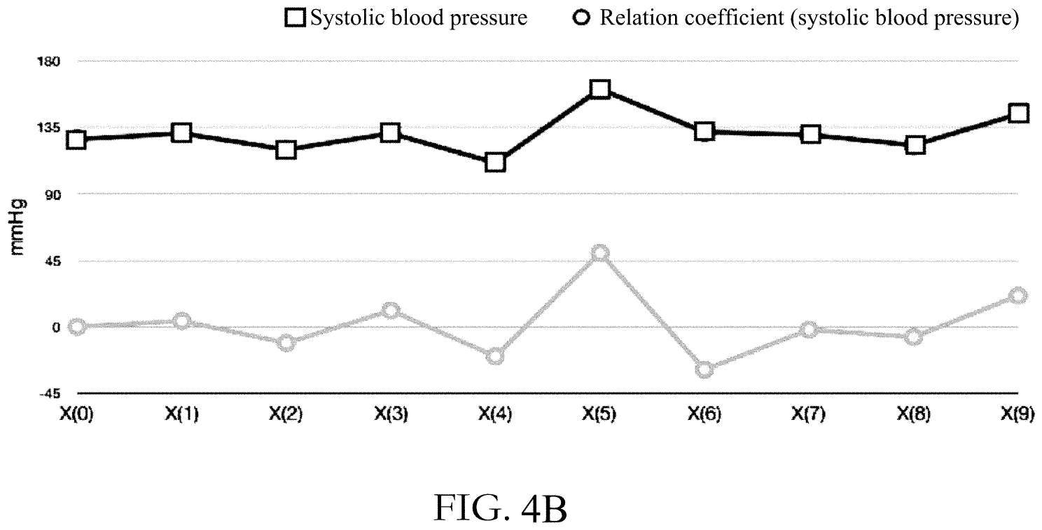

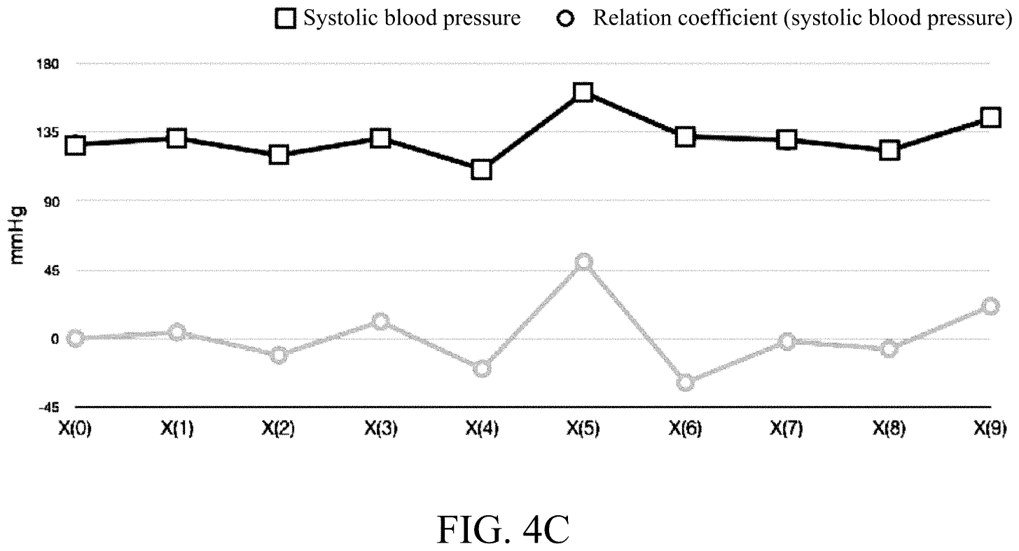

In one example, the feature processing program 134 further filters the feature sequence values according to the low blood pressure threshold, with reference to FIG. 4A. In this example, a prediction system 001 of a low blood pressure includes at least one data collection end 120 and a server 130. The architecture of hardware of the data collection end 120 and the server 130 is the same as in the previous example, so the description of the component configurations will not be repeated. In this example, the feature processing program 134 executes the flow as follows.

Step S410: A plurality of feature sequence values are obtained.

Step S420: Two feature sequence values are selected from the feature sequence values according to a time ratio relationship.

Step S430: A relation coefficient is calculated according to the selected two feature sequence values by a weighting process.

Step S440: It is determined whether the relation coefficient is more than a low blood pressure threshold.

Step S450: If the relation coefficient is more than a low blood pressure threshold, the two feature sequence values corresponding to the relation coefficient are assigned into an input group.

Step S460: To select the new feature sequence values and to assign the new feature sequence values into the input group are repeated until all the feature sequence values are traversed.

Step S470: If the relation coefficient is less than the low blood pressure threshold, a training result is obtained by substituting the input group into a low blood pressure prediction model.

The feature processing program 134 determines whether the relation coefficient is more than the low blood pressure threshold. If the relation coefficient is more than the low blood pressure threshold, the feature processing program 134 adds the two feature sequence values corresponding to the relation coefficient to the input group 136. The feature processing program 134 repeats to select the feature sequence values and to calculate the relation coefficient until all the feature sequence values are traversed. The low blood pressure threshold is determined according to intradialytic hypotension (IDH) criteria under different models. For the low blood pressure threshold, reference can be made to the following table, which shows a low blood pressure threshold correspondingly adopted by various models for determining a low blood pressure:

TABLE 1
Schematic table of a low blood pressure threshold

| | Systolic blood pressure | Mean arterial |
|---|---|---|
| | (Systole, mmHg) | pressure |
| IDH-1 | X(n) ≤ 900 | |
| IDH-2 | X(l) − X(n + l) ≥ 20 | X(l) − X(n + l) ≥ 10 |
| IDH-3 | X(n) − X(n + l) ≥ 20 | X(n) − X(n + l) ≥ 10 |
| IDH-4 | X(n) ≤ 90 & X(n) ≤ 100 | |
| IDH-5 | X(n) − X(n + l) ≥ y, y∈[20, 25, 30] | |
| IDH-6 | X(n) − X(n + l) ≥ X(n) * y, | |
| | y∈[0.2, 0.25, 0.3] | |

In the column of a systolic blood pressure in Table 1, the systolic blood pressure is used as the criterion for various low blood pressure determinations. The mean arterial pressure is calculated based on the systolic blood pressure and the diastolic blood pressure. The mean arterial pressure MAP=(systolic blood pressure-diastolic blood pressure)/3+diastolic blood pressure. IDH-2 and IDH-3 need to meet any set criterion of the systolic blood pressure and the mean arterial pressure to determine whether the low blood pressure threshold is satisfied. In other words, in IDH-2 and IDH-3, as long as the criterion of either the systolic blood pressure or the mean arterial pressure is met, the low blood pressure threshold is satisfied.

Therefore, the feature sequence value is a set of multiple parameters (a systolic blood pressure and a diastolic blood pressure). However, IDH-1, IDH-4, IDH-5 and IDH-6 use the systolic blood pressure as the single parameter. Those skilled in the art can construct other criteria for determining a low blood pressure thresholds according to different feature sequence values. For example: when the weighting process is the quotient operation, the low blood pressure threshold may be the quotient of various systolic blood pressures or other combinations.

In the process of repeating to select the feature sequence values, the feature processing program 134 may select the feature sequence values of the next round from the feature sequence values of the current round in a recursive manner. In this example, the increment of time is used as the manner for selecting the feature sequence values. In other words, the feature processing program 134 selects the last feature sequence value in the current round as the feature sequence value of the next round.

The feature processing program 134 determines whether the relation coefficient is more than or less than the low blood pressure threshold. If the relation coefficient is more than the low blood pressure threshold, the feature processing program 134 adds the two feature sequence values corresponding to the relation coefficient to an input group 136. After the feature processing program 134 completes the update of the input group 136, the feature processing program 134 performs calculation and comparison on the relation coefficient of the next round. At the same sampling time point, the number of the relation coefficients is at least greater than or equal to one. The low blood pressure thresholds of Table 1 are taken as the example. The determination of the low blood pressure threshold of IDH-2 and IDH-3 needs to satisfy the criteria of both the systolic blood pressure and the mean arterial pressure. Therefore, the relation coefficient also includes the calculation result of the systolic blood pressure and the calculation result of the mean arterial pressure.

If the relation coefficient is less than the low blood pressure threshold, the feature processing program 134 stops the assignment of the feature sequence values of this round. The feature processing program 134 obtains a corresponding training result 136 by substituting the input group 136 into the low blood pressure prediction model 135.

For further description, the operation of this example is described below by taking 10 sets of feature sequence values obtained in the current cycle 310 as an example. However, in fact, the data collection end 120 can transmit the feature sequence values in a real time for the server 130 to calculate. First, the data receiving terminal obtains the feature sequence values of an object 110. The feature sequence values and the relation coefficients are shown in the table below:

TABLE 2
List of feature sequence values and relation coefficients

| | Feature sequence value | | | | | | | | | |
|---|---|---|---|---|---|---|---|---|---|---|
| | X(0) | X(1) | X(2) | X(3) | X(4) | X(5) | X(6) | X(7) | X(8) | X(9) |
| Systolic blood | 127 | 131 | 120 | 131 | ill | 161 | 132 | 130 | 123 | 144 |
| pressure | | | | | | | | | | |
| Mean arterial | 81 | 83 | 81 | 82 | 76 | 98 | 81 | 82 | 76 | 79 |
| pressure | | | | | | | | | | |
| Relation coefficient | 0 | 4 | −11 | 11 | −20 | 50 | −29 | −2 | −7 | 21 |
| (systolic blood | | | | | | | | | | |
| pressure) | | | | | | | | | | |
| Relation coefficient | 0 | 2 | −2 | 1 | −6 | 22 | −17 | 1 | −6 | 3 |
| (mean arterial | | | | | | | | | | |
| pressure) | | | | | | | | | | |

The feature processing program 134 receives 10 sets of feature sequence values X(0)-X(9) sampled at different time points. The feature processing program 134 selects the feature sequence values by taking the time ratio relationship of “1”. In this example, the feature sequence value is the systolic blood pressure and the mean arterial pressure. The feature processing program 134 selects two adjacent feature sequence values for the weighting process. In this example, the weighting process is described as the difference operation. The feature processing program 134 can obtain 8 relation coefficients as shown in Table 2. Since there is no data before the feature sequence value X(0), no corresponding relation coefficient is obtained for X(0). The feature processing program 134 takes IDH-3 as the criterion for determining a low blood pressure threshold.

With reference to FIG. 4B and FIG. 4C, FIG. 4B and FIG. 4C are broken line graphs of feature sequence values and relation coefficients corresponding to Table 2. From FIG. 4B, variations of the feature sequence value and the relation coefficient (a systolic blood pressure) can be known. From FIG. 4C, variations of the feature sequence value and the relation coefficient (a mean arterial pressure) can be known. When the relation coefficient is R(x), the feature processing program 134 determines that the low blood pressure threshold is satisfied. Therefore, the feature processing program 134 assigns the feature sequence values X(0)-X(9) and the relation coefficients R_{SBP}(1)-R_{SBP}(9) and R_{MAP}(1)-R_{MAP}(9) into the input group 136. The feature processing program 134 substitutes various parameters of the input group into the low blood pressure prediction model 135 for the low blood pressure prediction model 135 to learn and to obtain the corresponding training result 136.

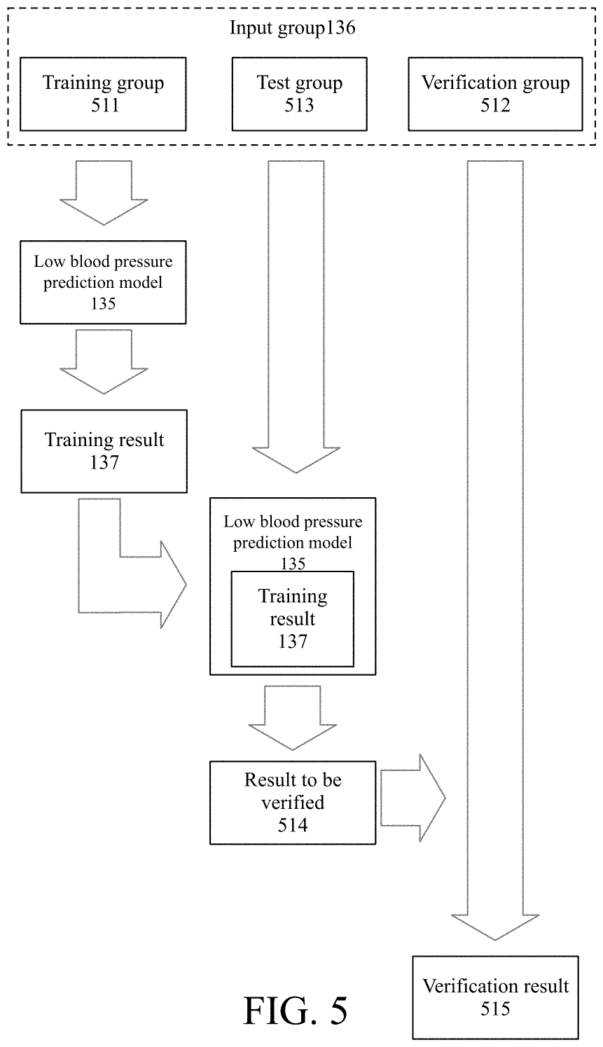

In one example, the feature processing program 134 further divides the input group 136 into a training group 511, a verification group 512 and a test group 513, with reference to FIG. 5. The training group 511 includes a plurality of sets of feature sequence values or relation coefficients. The verification group 512 includes a plurality of sets of feature sequence values or relation coefficients. The test group 513 includes a plurality of sets of feature sequence values or relation coefficients. The training group 511, the verification group 512 and the test group 513 may repeat the feature sequence values or the relation coefficients with the same contents. The processor 131 obtains a training result 136 by substituting the training group 511 into the low blood pressure prediction model 135. The processor 131 obtains a result to be verified 514 by substituting the test group 513 into the training result 136 and the low blood pressure prediction model 135. The processor 131 obtains a verification result 515 according to the verification group 512 and the result to be verified 514. In addition, the feature processing program 134 may also import feature sequence values obtained in other time periods to serve as the training group 511, the verification group 512 or the test group 513.

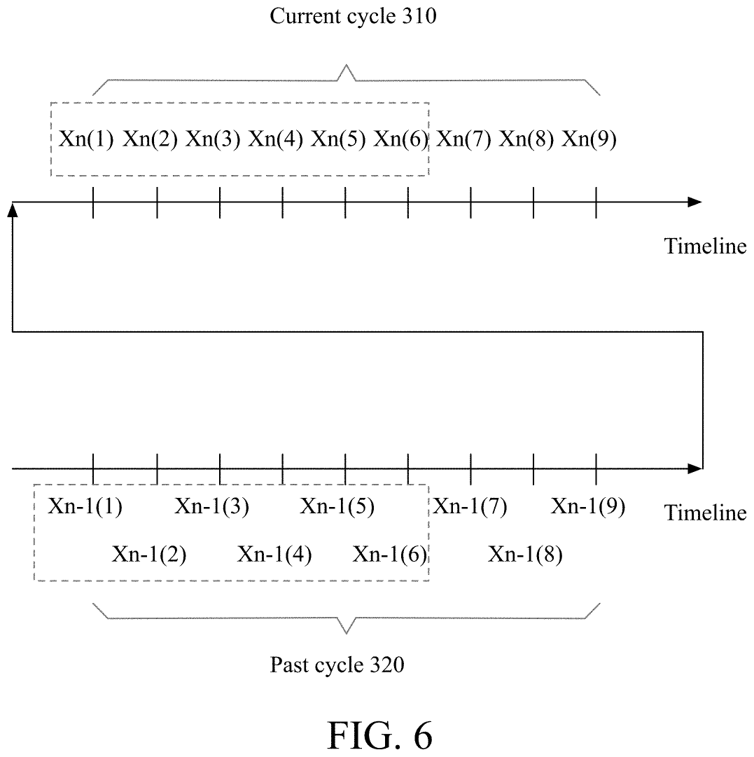

In one example, in addition to the current cycle 310, the feature processing program 134 further adds the relation processing of the feature sequence values of the past cycle 320. For architecture of the system 001 of this example, reference can be made to FIG. 1. In order to clearly describe the feature sequence values of the current cycle 310 and the past cycle 320, the feature sequence values of the current cycle 310 are also referred to as first eigenvalues, and the feature sequence values of the past cycle 320 are referred to as second eigenvalues. FIG. 6 is a schematic diagram showing distribution of the feature sequence values of the current cycle 310 and the past cycle 320 in this example.

The current cycle 310 and the past cycle 320 have a time corresponding relationship. The current obtainment period of the data collection end 120 is referred to as the current cycle 310, and the obtainment period relative to the current cycle 310 is referred to as the past cycle 320. There is a time corresponding relationship between the current cycle 310 and the past cycle 320. The time corresponding relationship is a specified time interval between the current cycle 310 and the past cycle 320. For example, if the time corresponding relationship is one week, then the interval between the current cycle 310 and the past cycle 320 is seven days. The feature sequence value is expressed as Xₙ(m). n is the cycle of the feature sequence value, and m is the number of sampling rounds. If the feature sequence value of the current cycle 310 is expressed as Xₙ(m) and the time corresponding relationship is one week, then the feature sequence value of the past cycle 320 is expressed as Xₙ₋₁(m). There is also a time corresponding relationship between the first eigenvalue and the second eigenvalue. The second eigenvalue may be stored in a storage unit 133 in advance.

The processor 131 obtains a corresponding first relation coefficient according to the first eigenvalue. The processor 131 obtains a corresponding second relation coefficient according to the second eigenvalue. The feature processing program 134 determines whether the first relation coefficient is more than the low blood pressure threshold. If the selected two first relation coefficients and the corresponding first relation coefficient are more than the low blood pressure threshold, then the feature processing program 134 assigns the two first eigenvalues corresponding to the first relation coefficient into the input group 136.

If the first relation coefficient is less than the low blood pressure threshold, the feature processing program 134 reads the corresponding second eigenvalue according to the first eigenvalue in the input group 136. The processor 131 adds the corresponding two first eigenvalues to the input group 136 according to the first relation coefficient. For example, the feature processing program 134 adds the first eigenvalues Xₙ(1)-Xₙ(m) to the input group 136, as shown by the dashed box in FIG. 6. The feature processing program 134 furthers reads the second eigenvalues Xₙ₋₁(1)-Xₙ₋₁(m) from the storage unit 133, as shown by the dashed box in FIG. 6. The feature processing program 134 also adds the second eigenvalues Xₙ₋₁(1)-Xₙ₋₁(m) to the input group 136.

The feature processing program 134 calculates the second relation coefficient for the second eigenvalue. The feature processing program 134 obtains the training result 136 by substituting the obtained first eigenvalue, second eigenvalue, first relation coefficient and second relation coefficient into the low blood pressure prediction model 135.

In the prediction method and system 001 of a low blood pressure, the relation coefficients are obtained according to the various feature sequence values of the object 110, and the feature sequence values and the relation coefficients are assigned by the feature processing program 134. The feature processing program 134 substitutes the assignment result into the low blood pressure prediction model 135, thereby obtaining the corresponding training result 136 of the object 110. The feature processing program 134 can more accurately predict blood pressure variations of the object 110 through the training result 136 and the low blood pressure prediction model 135.

What is claimed is:

1. A prediction method of a low blood pressure, comprising:
obtaining a plurality of feature sequence values;
selecting two of the feature sequence values from the feature sequence values according to a time ratio relationship;
calculating a relation coefficient according to the selected two feature sequence values by a weighting process;
repeating to select the new feature sequence values and the corresponding relation coefficient and to assign the new feature sequence values and the relation coefficient into the input group until the feature sequence values conforming to the time ratio relationship are traversed; and
obtaining a training result by substituting the input group into a low blood pressure prediction model.

2. The prediction method of a low blood pressure according to claim 1, wherein the step of obtaining the feature sequence values comprises:
setting a current cycle and a past cycle, wherein the current cycle and the past cycle have a time corresponding relationship;
using the feature sequence value obtained in the current cycle as a first eigenvalue; and
using the feature sequence value obtained in the past cycle as a second eigenvalue, wherein each of the first eigenvalues corresponds to the second eigenvalue according to the time corresponding relationship.

3. The prediction method of a low blood pressure according to claim 2, wherein the step of calculating the relation coefficient by the weighting process comprises:
obtaining a corresponding first relation coefficient according to the first eigenvalues; and
obtaining a corresponding second relation coefficient according to the second eigenvalues.

4. The prediction method of a low blood pressure according to claim 3, wherein the step of assigning the relation coefficient into the input group comprises:
adding the corresponding two of the first eigenvalues to the input group according to the first relation coefficient;
obtaining the second relation coefficient corresponding to the selected first relation coefficient according to the time corresponding relationship; and
adding the corresponding two of the second eigenvalues obtained according to the second relation coefficient to the input group.

5. The prediction method of a low blood pressure according to claim 1, wherein the step of obtaining the training result by substituting the input group into the low blood pressure prediction model comprises:
dividing the feature sequence values of the input group into a training group, a verification group and a test group; and
obtaining the training result corresponding to the training group by inputting the training group into the low blood pressure prediction model.

6. The prediction method of a low blood pressure according to claim 5, wherein the step of obtaining the training result corresponding to the training group by inputting the training group into the low blood pressure prediction model comprises:
obtaining a result to be verified by substituting the test group into the training result and the low blood pressure prediction model; and
obtaining a verification result according to the verification group and the result to be verified.

7. The prediction method of a low blood pressure according to claim 1, wherein the step of repeating to select the new feature sequence values and the corresponding relation coefficient and to assign the new feature sequence values and the relation coefficient into the input group comprises:
if the relation coefficient is more than a low blood pressure threshold, assigning the relation coefficient and the two feature sequence values corresponding to the relation coefficient into an input group; and
repeating to select the new feature sequence values and to assign the new feature sequence values into the input group until all the feature sequence values are traversed.

8. The prediction method of a low blood pressure according to claim 1, wherein the feature sequence values comprise heart rate variation coefficient, heart rate mean, blood oxygen variation coefficient, blood oxygen mean, diastolic blood pressure, mean arterial pressure, pulse, systolic blood pressure, pulse pressure, blood flow velocity, cumulative exchange blood volume, loop arterial pressure, blood temperature, bicarbonate concentration, electrical conductivity, dialysate flow velocity, anticoagulant maintenance dose, sodium ion, target sodium ion concentration, artificial kidney transmembrane pressure, machine set temperature, dehydration rate, dehydration time, current total dehydration, loop venous pressure or combinations thereof.

9. The prediction method of a low blood pressure according to claim 1, wherein the weighting process comprises a difference operation, a quotient operation, a multinomial coefficient operation, a trend calculation, an autoregressive model or a moving average model; and the low blood pressure prediction model comprises a LightGBM model, an Xgboost model, a Linear Regression model, a Random Forest model, a One Dimensional Convolutional Neural Network model, a Deep Neural Network model, a Long Short Term Memory Networks model or a Gated Recurrent Unit model.

10. A prediction system of a low blood pressure, comprising:
a data collection end, receiving a plurality of feature sequence values; and
a server, connected to the data collection end, wherein the server has a processor, a communication unit and a storage unit, the communication unit is connected to the data collection end, the communication unit transmits the feature sequence values, the storage unit stores a feature processing program and a low blood pressure prediction model, the processor executes the feature processing program, the processor obtains the feature sequence values through the transmission unit, the processor selects two of the feature sequence values according to a time ratio relationship, and the processor calculates a relation coefficient according to the selected two feature sequence values by a weighting process; the processor repeats to select the new feature sequence values and the corresponding relation coefficient and to assign the new feature sequence values and the relation coefficient into the input group until the feature sequence values conforming to the time ratio relationship are traversed; and the processor obtains a training result by substituting the input group into a low blood pressure prediction model.
